(19) United States
(12) Patent Application Publication
(10) Pub. No.: US 2011/0201716 A1
(43) Pub. Date: Aug. 18, 2011

(54) POLYESTER POLYOLS BASED ON TEREPHTHALIC ACID

(75) Inventors: Lionel Gehinger, Schaffhouse-pres-Seltz (FR); Gunnar Kampf, Stemwede-Haldem (DE); Gerlinde Tischer, Ruhland (DE)

(73) Assignee: BASF SE, Ludwigshafen (DE)

(21) Appl. No.: 13/124,217

(22) PCT Filed: Oct. 13, 2009
(86) PCT No.: PCT/EP09/63358

§ 371 Date: Apr. 14, 2011

(30) Foreign Application Priority Data
Oct. 15, 2008 (EP) 08166708.1

(51) Int. Cl. C08G 18/34; C07C 69/76; C08J 9/06

(52) U.S. Cl. 521/172; 560/64

(57) ABSTRACT

Polyester polyol comprising the esterification product of
a) from 10 to 70 mol % of a dicarboxylic acid composition comprising
   a1) from 50 to 100 mol % of a material based on terephthalic acid, selected from among terephthalic acid, dimethyl terephthalate, polyalkylene terephthalate and mixtures thereof,
   a2) from 0 to 50 mol % of phthalic acid, phthalic anhydride or isophthalic acid,
   a3) from 0 to 50 mol % of one or more dicarboxylic acids,
b) from 2 to 30 mol % of one or more fatty acids and/or fatty acid derivatives and/or benzoic acid,
c) from 10 to 70 mol % of one or more aliphatic or cycloaliphatic diols having from 2 to 18 carbon atoms or alkoxylates thereof,
d) from 2 to 50 mol % of a higher-functional polyol selected from the group consisting of glycerol, alkoxylated glycerol, trimethylolpropane, alkoxylated trimethylolpropane, pentaerythritol and alkoxylated pentaerythritol,
   wherein at least 200 mmol, preferably at least 500 mmol and particularly preferably at least 800 mmol, of polyols d) having an OH functionality of 2.9 are reacted per kg of polyester polyol.

The invention relates to polyester polyols based on terephthalic acid and their use for producing rigid polyurethane foams.

The production of rigid polyurethane foams by reacting organic or modified organic diisocyanates or polyisocyanates with relatively high molecular weight compounds having at least two reactive hydrogen atoms, in particular with polyether polyols from alkylene oxide polymerization or polyester polyols from the polycondensation of alcohols with dicarboxylic acids, in the presence of polyurethane catalysts, chain extenders and/or crosslinkers, blowing agents and further auxiliaries and additives is known and is described in numerous patent and literature publications.

Mention may be made by way of example of the Kunststoffhandbuch, Volume VII, Polyurethane, Carl-Hanser-Verlag, Munich, 1st Edition 1966, edited by Dr. R. Vieweg and Dr. A. Höchtlen, and 2nd Edition 1983 and 3rd Edition 1993, edited by Dr. G. Oertel. Appropriate selection of the formative components and their ratios enables polyurethane foams having very good mechanical properties to be produced.

When polyester polyols are used, it is usual to employ polycondensates of aromatic and/or aliphatic dicarboxylic acids and alkanediols and/or alkanetriols or ether diols. However, it is also possible to process polyester scrap, in particular polyethylene terephthalate (PET) or polybutylene terephthalate (PBT) scrap. A whole series of processes are known and have been described for this purpose. Some processes are based on the conversion of the polyester into a diester of terephthalic acid, e.g. dimethyl terephthalate. DE-A 1003714 and U.S. Pat. No. 5,051,528 describe such transesterifications using methanol and transesterification catalysts.

It is also known that esters based on terephthalic acid are superior in terms of the burning behavior to esters based on phthalic acid. However, the high tendency to crystallize and thus low storage stability of esters based on terephthalic acid is a disadvantage.

To increase the storage stability of polyester polyols based on terephthalic acid which tend to crystallize rapidly, it is usual to add aliphatic dicarboxylic acids. However, these have an adverse effect on the burning behavior (flame resistance) of the polyurethane foams produced therewith.

It is an object of the invention to provide polyester polyols which are based on terephthalic acid or terephthalic acid derivatives and have an improved storage stability. A further object of the invention is to provide polyester polyols having improved storage stability which give polyurethane foams having an improved burning behavior.

This object is achieved by a polyester polyol comprising the esterification product of
a) from 10 to 70 mol %, preferably from 20 to 70 mol % and particularly preferably from 25 to 50 mol %, of a dicarboxylic acid composition comprising
   a1) from 50 to 100 mol % of a material based on terephthalic acid, selected from among terephthalic acid, dimethyl terephthalate, polyalkylene terephthalate and mixtures thereof,
   a2) from 0 to 50 mol % of phthalic acid, phthalic anhydride or isophthalic acid,
   a3) from 0 to 50 mol % of one or more dicarboxylic acids,
b) from 2 to 30 mol %, preferably from 3 to 20 mol %, particularly preferably from 4 to 15 mol %, of fatty acids, one or more fatty acid derivatives and/or benzoic acid,
c) from 10 to 70 mol %, preferably from 20 to 60 mol %, particularly preferably from 25 to 55 mol %, of one or more aliphatic or cycloaliphatic diols having from 2 to 18 carbon atoms or alkoxylates thereof,
d) from 2 to 50 mol %, preferably from 2 to 40 mol %, particularly preferably from 2 to 35 mol %, of a higher-functional polyol selected from the group consisting of glycerol, alkoxylated glycerol, trimethylolpropane, alkoxylated trimethylolpropane, pentaerythritol and alkoxylated pentaerythritol,
   wherein at least 200 mmol, preferably at least 500 mmol and particularly preferably at least 800 mmol, of polyols d) having an OH functionality of 2.9 are reacted per kg of polyester polyol.

The dicarboxylic acid composition a) preferably comprises more than 50 mol % of the material a1), based on terephthalic acid, preferably more than 75 mol % and particularly preferably 100 mol % of the material a1) based on terephthalic acid.

The aliphatic diol is preferably selected from the group consisting of ethylene glycol, diethylene glycol, polyethylene glycol, propylene glycol, 1,3-propanediol, 1,4-butanediol, 1,5-pentanediol, 1,6-hexanediol, 2-methyl-1,3-propanediol, 3-methyl-1,5-pentanediol and alkoxylates thereof, in particular ethoxylates thereof. In particular, the aliphatic diol is diethylene glycol.

The fatty acid or the fatty acid derivative b) is preferably a fatty acid or a fatty acid derivative based on renewable raw materials and selected from the group consisting of castor oil, polyhydroxy fatty acids, ricinoleic acid, hydroxyl-modified oils, grapeseed oil, black cumin oil, pumpkin kernel oil, borage seed oil, soybean oil, wheat germ oil, rapeseed oil, sunflower oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, hydroxyl-modified fatty acids and fatty acid esters based on myristoleic acid, palmitoleic acid, oleic acid, vaccenic acid, petroselic acid, gadoleic acid, erucic acid, nervonic acid, linoleic acid, α- and γ-linolenic acid, stearidonic acid, arachidonic acid, timnodonic acid, clupanodonic acid and cervonic acid.

The esterification or transesterification is carried out under customary esterification or transesterification conditions. Here, the aromatic and aliphatic dicarboxylic acids or dicarboxylic esters and polyhydric alcohols are reacted in the absence of catalysts or preferably in the presence of esterification catalysts, advantageously in an atmosphere of inert gas, e.g. nitrogen, carbon monoxide, helium, argon, etc., in the melt at temperatures of from 150 to 260° C., preferably from 180 to 250° C., if appropriate under reduced pressure, with the low molecular weight alcohol liberated by the transesterification (for example methanol) being distilled off, preferably under reduced pressure. Possible esterification catalysts are, for example, iron, cadmium, cobalt, lead, zinc, antimony, magnesium, titanium and tin catalysts in the form of metals, metal oxides or metal salts. The transesterification can also be carried out in the presence of diluents and/or entrainers such as benzene, toluene, xylene or chlorobenzene in order to distill off the water of condensation as an azeotrope.

The invention also provides a process for producing rigid polyurethane foams by reacting
A) organic and/or modified organic diisocyanates and/or polyisocyanates with
B) the specific polyester polyols according to the invention, with the component B) being able to comprise up to 50% by weight of further polyester polyols,
C) if appropriate, polyetherols and/or further compounds having at least two groups which are reactive toward isocyanates and, if appropriate, chain extenders and/or crosslinkers,
D) blowing agents,
E) catalysts and, if appropriate,
F) further auxiliaries and/or additives,
G) flame retardants.

To produce the rigid polyurethane foams by the process of the invention, use is made of, in addition to the above-described specific polyester polyols, the formative components which are known per se, about which the following details may be provided.

Possible organic and/or modified organic polyisocyanates A) are the aliphatic, cycloaliphatic, araliphatic and preferably aromatic polyfunctional isocyanates known per se.

Specific examples are: alkylene diisocyanates having from 4 to 12 carbon atoms in the alkylene radical, e.g. dodecane 1,12-diisocyanate, 2-ethyltetramethylene 1,4-diisocyanate, 2-methylpentamethylene 1,5-diisocyanate, tetramethylene 1,4-diisocyanate, and preferably hexamethylene 1,6-diisocyanate; cycloaliphatic diisocyanates such as cyclohexane 1,3- and 1,4-diisocyanate and also any mixtures of these isomers, 1-isocyanato-3,3,5-trimethyl-5-isocyanatomethylcyclohexane (IPDI), hexahydrotolylene 2,4- and 2,6-diisocyanate and also the corresponding isomer mixtures, dicyclohexylmethane 4,4′-, 2,2′- and 2,4′-diisocyanate and also the corresponding isomer mixtures and preferably aromatic diisocyanates and polyisocyanates such as tolylene 2,4- and 2,6-diisocyanate and the corresponding isomer mixtures, diphenylmethane 4,4′-, 2,4′- and 2,2′-diisocyanate and the corresponding isomer mixtures, mixtures of diphenylmethane 4,4′- and 2,2′-diisocyanates, polyphenylpolymethylene polyisocyanates, mixtures of diphenylmethane 2,4′-, 2,4′- and 2,2′-diisocyanates and polyphenylpolymethylene polyisocyanates (crude MDI) and mixtures of crude MDI and tolylene diisocyanates. The organic diisocyanates and polyisocyanates can be used individually or in the form of their mixtures.

Preferred diisocyanates and polyisocyanates are tolylene diisocyanate (TDI), diphenylmethane diisocyanate (MDI) and in particular mixtures of diphenylmethane diisocyanate and polyphenylenepolymethylene polyisocyanates (polymeric MDI or PMDI).

Use is frequently also made of modified polyfunctional isocyanates, i.e. products which are obtained by chemical reaction of organic diisocyanates and/or polyisocyanates. Examples which may be mentioned are diisocyanates and/or polyisocyanates comprising ester, urea, biuret, allophanate, carbodiimide, isocyanurate, uretdione, carbamate and/or urethane groups.

Very particular preference is given to using polymeric MDI for producing rigid polyurethane foams.

In the prior art, it is sometimes customary to incorporate isocyanurate groups into the polyisocyanate. This is preferably carried out using catalysts which form isocyanurate groups, for example alkali metal salts either alone or in combination with tertiary amines. Isocyanurate formation leads to flame-resistant polyisocyanurate foams (PIR foams) which are preferably used in industrial rigid foam, for example in building and construction as insulation board or sandwich elements.

Suitable further polyester polyols can be prepared, for example, from organic dicarboxylic acids having from 2 to 12 carbon atoms, preferably aliphatic dicarboxylic acids having from 4 to 6 carbon atoms, and polyhydric alcohols, preferably diols, having from 2 to 12 carbon atoms, preferably from 2 to 6 carbon atoms. Possible dicarboxylic acids are, for example: succinic acid, glutaric acid, adipic acid, suberic acid, azelaic acid, sebacic acid, decanedicarboxylic acid, maleic acid, fumaric acid, phthalic acid, isophthalic acid and terephthalic acid. The dicarboxylic acids can be used either individually or in admixture with one another. It is also possible to use the corresponding dicarboxylic acid derivatives, e.g. dicarboxylic esters of alcohols having from 1 to 4 carbon atoms or dicarboxylic anhydrides, in place of the free dicarboxylic acids. Preference is given to using dicarboxylic acid mixtures of succinic, glutaric and adipic acid in weight ratios of, for example, 20-35:35-50:20-32 and in particular adipic acid. Examples of dihydric and polyhydric alcohols, in particular diols, are: ethanediol, diethylene glycol, 1,2- or 1,3-propanediol, dipropylene glycol, 1,4-butanediol, 1,5-pentanediol, 1,6-hexanediol, 1,10-decanediol, glycerol, trimethylolpropane and pentaerythritol. Preference is given to using ethanediol, diethylene glycol, 1,4-butanediol, 1,5-pentanediol, 1,6-hexanediol or mixtures of at least two of the diols mentioned, in particular mixtures of 1,4-butanediol, 1,5-pentanediol and 1,6-hexanediol. It is also possible to use polyester polyols derived from lactones, e.g. ε-caprolactone, or hydroxycarboxylic acids, e.g. ω-hydroxycaproic acid.

To prepare the polyester polyols, the organic, e.g. aromatic and preferably aliphatic, polycarboxylic acids and/or derivatives and polyhydric alcohols can be polycondensed in the absence of catalysts or preferably in the presence of esterification catalysts, advantageously in an atmosphere of inert gas, e.g. nitrogen, carbon monoxide, helium, argon, etc., in the melt at temperatures of from 150 to 260° C., preferably from 180 to 250° C., if appropriate under reduced pressure, to the desired acid number which is advantageously less than 10, preferably less than 2. In a preferred embodiment, the esterification mixture is polycondensed at the abovementioned temperatures to an acid number of from 80 to 20, preferably from 40 to 20, under atmospheric pressure and subsequently under a pressure of less than 500 mbar, preferably from 40 to 200 mbar. Possible esterification catalysts are, for example, iron, cadmium, cobalt, lead, zinc, antimony, magnesium, titanium and tin catalysts in the form of metals, metal oxides or metal salts. However, the polycondensation can also be carried out in the liquid phase in the presence of diluents and/or entrainers such as benzene, toluene, xylene or chlorobenzene to distill off the water of condensation as an azeotrope.

To prepare the polyester polyols, the organic polycarboxylic acids and/or derivatives and polyhydric alcohols are advantageously polycondensed in a molar ratio of 1:1-2.1, preferably 1:1.05-1.9.

The polyester polyols obtained preferably have a functionality of from 2 to 4, in particular from 2 to 3, and a molecular weight of from 300 to 3000, preferably from 400 to 1000 and in particular from 450 to 800.

It is also possible to make concomitant use of polyether polyols which are prepared by known methods, for example from one or more alkylene oxides having from 2 to 4 carbon atoms in the alkylene radical by anionic polymerization using alkali metal hydroxides, e.g. sodium or potassium hydroxide, or alkali metal alkoxides, e.g. sodium methoxide, sodium or potassium ethoxide or potassium isopropoxide, as catalysts with addition of at least one starter molecule comprising from 2 to 8, preferably from 2 to 6, reactive hydrogen atoms, or by cationic polymerization using Lewis acids, e.g. antimony pentachloride, boron fluoride etherate, etc., or bleaching earth, as catalysts.

Suitable alkylene oxides are, for example, tetrahydrofuran, 1,3-propylene oxide, 1,2- or 2,3-butylene oxide, styrene oxide and preferably ethylene oxide and 1,2-propylene oxide. The alkylene oxides can be used individually, alternately in succession or as mixtures. Preferred alkylene oxides are propylene oxide and ethylene oxide, with particular preference being given to ethylene oxide.

Possible starter molecules are, for example: water, organic dicarboxylic acids, such as succinic acid, adipic acid, phthalic acid and terephthalic acid, aliphatic and aromatic, unsubstituted or N-monoalkyl-, N,N-dialkyl- and N,N′-dialkyl-substituted diamines having from 1 to 4 carbon atoms in the alkyl radical, e.g. unsubstituted or monoalkyl- and dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexamethylenediamine, phenylenediamines, 2,3-, 2,4- and 2,6-toluenediamine and 4,4′-, 2,4′- and 2,2′-diaminodiphenylmethane.

Further possible starter molecules are: alkanolamines such as ethanolamine, N-methylethanolamine and N-ethylethanolamine, dialkanolamines, such as diethanolamine, N-methyldiethanolamine and N-ethyldiethanolamine and trialkanolamines, such as triethanolamine, and ammonia. Preference is given to using dihydric or polyhydric alcohols such as ethanediol, 1,2- and 1,3-propanediol, diethylene glycol, dipropylene glycol, 1,4-butanediol, 1,6-hexanediol, glycerol, trimethylolpropane, pentaerythritol, ethylenediamine, sorbitol and sucrose.

The polyether polyols, preferably polyoxypropylene and polyoxypropylenepolyoxyethylene polyols, have a functionality of preferably from 2 to 6 and in particular from 2 to 5 and molecular weights of from 300 to 3000, preferably from 300 to 2000 and in particular from 400 to 1000.

Further suitable polyether polyols are polymer-modified polyether polyols, preferably graft polyether polyols, in particular those based on styrene and/or acrylonitrile which are prepared by in-situ polymerization of acrylonitrile, styrene or preferably mixtures of styrene and acrylonitrile, e.g. in a weight ratio of from 90:10 to 10:90, preferably from 70:30 to 30:70, advantageously in the abovementioned polyether polyols using methods analogous to those described in the German patent texts 11 11 394, 12 22 669 (U.S. Pat. Nos. 3,304,273, 3,383,351, 3,523,093), 11 52 536 (GB 10 40 452) and 11 52 537 (GB 987,618), and also polyether polyol dispersions which comprise, for example, polyureas, polyhydrazides, polyurethanes comprising bound tert-amino groups and/or melamine as disperse phase, usually in an amount of from 1 to 50% by weight, preferably from 2 to 25% by weight, and are described, for example, in EP-B 011 752 (U.S. Pat. No. 4,304,708), U.S. Pat. No. 4,374,209 and DE-A,32 31 497.

Like the polyester polyols, the polyether polyols can be used individually or in the form of mixtures. They can also be mixed with the graft polyether polyols or polyester polyols and with the hydroxyl-comprising polyesteramides, polyacetals, polycarbonates and/or polyether polyamines.

Possible hydroxyl-comprising polyacetals are, for example, the compounds which can be prepared from glycols such as diethylene glycol, triethylene glycol, 4,4′-dihydroxyethoxydiphenyldimethylmethane, hexanediol and formaldehyde. Suitable polyacetals can also be prepared by polymerization of cyclic acetals.

Possible hydroxyl-comprising polycarbonates are those of the type known per se which can be prepared, for example, by reacting diols such as 1,3-propanediol, 1,4-butanediol and/or 1,6-hexanediol, diethylene glycol, triethylene glycol or tetraethylene glycol with diaryl carbonates, e.g. diphenyl carbonate, or phosgene.

The polyesteramides include, for example, the predominantly linear condensates obtained from polybasic, saturated and/or unsaturated carboxylic acids or anhydrides thereof and polyhydric saturated and/or unsaturated amino alcohols or mixtures of polyhydric alcohols and amino alcohols and/or polyamines.

Suitable polyether polyamines can be prepared from the abovementioned polyether polyols by known methods. Mention may be made by way of example of the cyanoalkylation of polyoxyalkylene polyols and subsequent hydrogenation of the nitrile formed (U.S. Pat. No. 3,267,050) or the partial or complete amination of polyoxyalkylene polyols with amines or ammonia in the presence of hydrogen and catalysts (DE 12 15 373).

The rigid polyurethane foams can be produced using chain extenders and/or crosslinkers C). However, the addition of chain extenders, crosslinkers or, if appropriate, mixtures thereof can prove to be advantageous for modifying the mechanical properties, e.g. the hardness. As chain extenders and/or crosslinkers, use is made of diols and/or triols having molecular weights of less than 400, preferably from 60 to 300. Possibilities are, for example, aliphatic, cycloaliphatic and/or araliphatic diols having from 2 to 14, preferably from 4 to 10 carbon atoms, e.g. ethylene glycol, 1,3-propanediol, 1,10-decanediol, o-, m-, p-dihydroxycyclohexane, diethylene glycol, dipropylene glycol and preferably 1,4-butanediol, 1,6-hexanediol and bis(2-hydroxyethyl)hydroquinone, triols such as 1,2,4-, 1,3,5-trihydroxycyclohexane, glycerol and trimethylolpropane and low molecular weight hydroxyl-comprising polyalkylene oxides based on ethylene oxide and/or 1,2-propylene oxide and the abovementioned diols and/or triols as starter molecules.

Possible further compounds C) having at least two groups which are reactive toward isocyanate, i.e. having at least two hydrogen atoms which are reactive toward isocyanate groups, are in particular those which have two or more reactive groups selected from among OH groups, SH groups, NH groups, NH₂ groups and CH-acid groups, e.g. β-diketo groups.

If chain extenders, crosslinkers or mixtures thereof are employed for producing the rigid polyurethane foams, they are advantageously used in an amount of from 0 to 20% by weight, preferably from 0.5 to 5% by weight, based on the weight of the component B).

Blowing agents D) which are used for producing the rigid polyurethane foams include preferably water, formic acid and mixtures thereof. These react with isocyanate groups to form carbon dioxide and in the case of formic acid carbon dioxide and carbon monoxide. In addition, physical blowing agents such as low-boiling hydrocarbons can be used. Suitable physical blowing agents are liquids which are inert towards the organic, modified or nonmodified polyisocyanates and have boiling points below 100° C., preferably below 50° C., at atmospheric pressure, so that they vaporize under the conditions of the exothermic polyaddition reaction. Examples of such liquids which can preferably be used are alkanes such as heptane, hexane, n-pentane and isopentane, preferably industrial mixtures of n-pentane and isopentane, n-butane and isobutane and propane, cycloalkanes such as cyclopentane and/or cyclohexane, ethers such as furan, dimethyl ether and diethyl ether, ketones such as acetone and methyl ethyl ketone, alkyl carboxylates such as methyl formate, dimethyl oxalate and ethyl acetate and halogenated hydrocarbons such as methylene chloride, dichloromonofluoromethane, difluoromethane, trifluoromethane, difluoroethane, tetrafluoroethane, chlorodifluoroethanes, 1,1-dichloro-2,2,2-trifluoroethane, 2,2-dichloro-2-fluoroethane and heptafluoropropane. Mixtures of these low-boiling liquids with one another and/or with other substituted or unsubstituted hydrocarbons can also be used. Organic carboxylic acids such as formic acid, acetic acid, oxalic acid, ricinoleic acid and carboxyl-comprising compounds are also suitable.

Preference is given to using water, formic acid, chlorodifluoromethane, chlorodifluoroethanes, dichlorofluoroethanes, pentane mixtures, cyclohexane and mixtures of at least two of these blowing agents, e.g. mixtures of water and cyclohexane, mixtures of chlorodifluoromethane and 1-chloro-2,2-difluoroethane and optionally water.

The blowing agents are either completely or partly dissolved in the polyol component (i.e. B+C+E+F+G) or are introduced via a static mixer immediately before foaming of the polyol component. It is usual for water or formic acid to be fully or partially dissolved in the polyol component and the physical blowing agent (for example pentane) and, if appropriate, the remainder of the chemical blowing agent to be introduced “on-line”.

The amount of blowing agent or blowing agent mixture used is from 1 to 45% by weight, preferably from 1 to 30% by weight, particularly preferably from 1.5 to 20% by weight, in each case based on the sum of the components B) to G).

If water serves as blowing agent, it is preferably added to the formative component B) in an amount of from 0.2 to 5% by weight, based on the formative component B). The addition of water can be combined with the use of the other blowing agents described.

Catalysts E) used for producing the rigid polyurethane foams are, in particular, compounds which strongly accelerate the reaction of the compounds comprising reactive hydrogen atoms, in particular hydroxyl groups, of component B) and, if used, C) with the organic, modified or nonmodified polyisocyanates A).

It is advantageous to use basic polyurethane catalysts, for example tertiary amines such as triethylamine, tributylamine, dimethylbenzylamine, dicyclohexylmethylamine, dimethylcyclohexylamine, bis(N,N-dimethylaminoethyl) ether, bis(dimethylaminopropyl)urea, N-methylmorpholine or N-ethylmorpholine, N-cyclohexylmorpholine, N,N,N′,N′-tetramethylethylenediamine, N,N,N′,N′-tetramethylbutanediamine,

N,N,N′,N′-tetramethylhexane-1,6-diamine, pentamethyldiethylenetriamine, dimethylpiperazine, N-dimethylaminoethylpiperidine, 1,2-dimethylimidazole, 1-azabicyclo[2.2.0]octane, 1,4-diazabicyclo[2.2.2]octane (Dabco) and alkanolamine compounds, such as triethanolamine, triisopropanolamine, N-methyldiethanolamine and N-ethyldiethanolamine, dimethylaminoethanol, 2-(N,N-dimethylaminoethoxy)ethanol, N,N′,N″-tris(dialkylaminoalkyl)hexahydrotriazines, e.g. N,N′,N″-tris(dimethylaminopropyl)-s-hexahydrotriazine, and triethylenediamine. However, metal salts such as iron(II) chloride, zinc chloride, lead octoate and preferably tin salts such as tin dioctoate, tin diethylhexoate and dibutyltin dilaurate and also, in particular, mixtures of tertiary amines and organic tin salts are also suitable.

Further possible catalysts are: amidines such as 2,3-dimethyl-3,4,5,6-tetrahydropyrimidine, tetraalkylammonium hydroxides such as tetramethylammonium hydroxide, alkali metal hydroxides such as sodium hydroxide and alkali metal alkoxides such as sodium methoxide and potassium isopropoxide and also alkali metal salts of long-chain fatty acids having from 10 to 20 carbon atoms and, if appropriate, lateral OH groups. Preference is given to using from 0.001 to 5% by weight, in particular from 0.05 to 2% by weight, of catalyst or catalyst combination, based on the weight of the component B). It is also possible to allow the reactions to proceed without catalysis. In this case, the catalytic activity of amine-initiated polyols is exploited. In the prior art, it is sometimes customary to incorporate isocyanurate groups into the polyisocyanate. This is preferably carried out using catalysts which form isocyanurate groups, for example ammonium salts or alkali metal salts either alone or in combination with tertiary amines. Isocyanurate formation leads to flame-resistant PIR foams which are preferably used in industrial rigid foam, for example in building and construction as insulation boards or sandwich elements.

Further information regarding the abovementioned and further starting materials may be found in the technical literature, for example Kunststoffhandbuch, Volume VII, Polyurethane, Carl Hanser Verlag Munich, Vienna, 1st, 2nd and 3rd Editions 1966, 1983 and 1993.

If appropriate, further auxiliaries and/or additives F) can be added to the reaction mixture for producing the rigid polyurethane foams. Mention may be made of, for example, surface-active substances, foam stabilizers, cell regulators, fillers, dyes, pigments, flame retardants, hydrolysis inhibitors, fungistatic and bacteriostatic substances.

Possible surface-active substances are, for example, compounds which serve to aid homogenization of the starting materials and may also be suitable for regulating the cell structure of the polymers. Mention may be made of, for example, emulsifiers such as the sodium salts of castor oil sulfates or of fatty acids and also salts of fatty acids with amines, e.g. diethylamine oleate, diethanolamine stearate, diethanolamine ricinoleate, salts of sulfonic acids, e.g. alkali metal or ammonium salts of dodecylbenzenesulfonic or dinaphthylmethanedisulfonic acid and ricinoleic acid; foam stabilizers such as siloxane-oxyalkylene copolymers and other organopolysiloxanes, ethoxylated alkylphenols, ethoxylated fatty alcohols, paraffin oils, castor oil esters or ricinoleic esters, Turkey red oil and peanut oil, and cell regulators such as paraffins, fatty alcohols and dimethylpolysiloxanes. The above-described oligomeric acrylates having polyoxyalkylene and fluoroalkane radicals as side groups are also suitable for improving the emulsifying action, the cell structure and/or for stabilizing the foam. The surface-active substances are usually employed in amounts of from 0.01 to 10% by weight, based on 100% by weight of the component B).

For the purposes of the present invention, fillers, in particular reinforcing fillers, are the customary organic and inorganic fillers, reinforcing materials, weighting agents, agents for improving the abrasion behavior in paints, coating compositions, etc., which are known per se. Specific examples are: inorganic fillers such as siliceous minerals, for example sheet silicates such as antigorite, serpentine, hornblendes, amphiboles, chrisotile and talc, metal oxides such as kaolin, aluminum oxides, titanium oxides and iron oxides, metal salts, such as chalk, barite and inorganic pigments such as cadmium sulfide and zinc sulfide and also glass, etc. Preference is given to using kaolin (china clay), aluminum silicate and coprecipitates of barium sulfate and aluminum silicate and also natural and synthetic fibrous minerals such as wollastonite, metal fibers and in particular glass fibers of various length, which may be coated with a size. Possible organic fillers are, for example: carbon, melamine, rosin, cyclopentadienyl resins and graft polymers and also cellulose fibers, polyamide, polyacrylonitrile, polyurethane, polyester fibers based on aromatic and/or aliphatic dicarboxylic esters and in particular carbon fibers.

The inorganic and organic fillers can be used individually or as mixtures and are advantageously added to the reaction mixture in amounts of from 0.5 to 50% by weight, preferably from 1 to 40% by weight, based on the weight of the components A) to C), although the content of mats, nonwovens and woven fabrics of natural and synthetic fibers can reach values of up to 80% by weight.

As flame retardants G), it is generally possible to use the flame retardants known from the prior art. Suitable flame retardants are, for example, unincorporatable brominated substances, brominated esters, brominated ethers (Ixol) or brominated alcohols such as dibromoneopentyl alcohol, tribromoneopentyl alcohol and PHT-4-diol and also chlorinated phosphates such as tris(2-chloroethyl) phosphate, tris(2-chloropropyl) phosphate, tris(1,3-dichloropropyl) phosphate, tricresyl phosphate, tris(2,3-dibromopropyl) phosphate, tetrakis(2-chloroethyl) ethylenediphosphate, dimethyl methanephosphonate, diethyl diethanolaminomethylphosphonate and also commercial halogen-comprising flame retardant polyols. As further liquid flame retardants, it is possible to use phosphates or phosphonates, e.g. diethyl ethanephosphonate (DEEP), triethylphosphate (TEP), dimethyl propylphosphonate (DMPP), diphenyl cresyl phosphate (DPK) and others.

Apart from the abovementioned flame retardants, it is possible to use inorganic or organic flame retardants such as red phosphorus, preparations comprising red phosphorus, aluminum oxide hydrate, antimony trioxide, arsenic oxide, ammonium polyphosphate and calcium sulfate, expandable graphite or cyanuric acid derivatives such as melamine, or mixtures of at least two flame retardants, e.g. ammonium polyphosphates and melamine and, if appropriate, maize starch or ammonium polyphosphate, melamine and expandable graphite and/or aromatic or nonaromatic polyesters for making the rigid polyurethane foams flame resistant.

In general, it has been found to be advantageous to use from 5 to 150% by weight, preferably from 10 to 100% by weight, of the flame retardants mentioned, based on the component B).

Further information regarding the abovementioned other customary auxiliaries and additives may be found in the technical literature, for example the monograph by J. H. Saunders and K. C. Frisch “High Polymers” Volume XVI, Polyurethanes, Parts 1 and 2, Interscience Publishers 1962 and 1964, or Kunststoff-Handbuch, Polyurethane, Volume VII, Hanser-Verlag, Munich, Vienna, 1st and 2nd Editions, 1966 and 1983.

To produce the rigid polyurethane foams of the invention, the organic and/or modified organic polyisocyanates A), the specific polyester polyols B) and, if appropriate, polyetherol and/or further compounds having at least two groups which are reactive toward isocyanates and, if appropriate, chain extenders and/or crosslinkers C) are reacted in such amounts that the equivalence ratio of NCO groups of the polyisocyanates A) to the sum of the reactive hydrogen atoms of the components B) and, if used, C) and D) to G) is 1-6:1, preferably 1.1-5:1 and in particular 1.2-3.5:1.

The rigid polyurethane foams are advantageously produced by the one-shot process, for example by means of the high-pressure or low-pressure technique, in open or closed molds, for example metallic molds. Continuous application of the reaction mixture to suitable conveyor belts for producing panels is also customary.

The starting components are mixed at a temperature of from 15 to 90° C., preferably from 20 to 60° C. and in particular from 20 to 35° C., and introduced into the open mold or, if appropriate under elevated pressure, into the closed mold or, in the case of a continuous workstation, applied to a belt which accommodates the reaction mixture. Mixing can, as indicated above, be carried out mechanically by means of a stirrer or a stirring screw. The mold temperature is advantageously from 20 to 110° C., preferably from 30 to 70° C. and in particular from 40 to 60° C.

The rigid polyurethane foams produced by the process of the invention have a density of from 15 to 300 g/l, preferably from 20 to 100 g/l and in particular from 25 to 60 g/l.

The invention is illustrated by the following examples.

### EXAMPLES

Various polyesterols were prepared:

### General Method

The dicarboxylic acid, the aliphatic or cycloaliphatic diol or alkoxylates thereof and the higher-functional polyol were introduced into a 4 liter round-bottom flask equipped with a mechanical stirrer, a thermometer and a distillation column and also a nitrogen inlet tube. After addition of 40 ppm of titanium tetrabutylate as catalyst, the mixture is stirred and heated to 240° C., with the water liberated being distilled off continuously. The reaction is carried out at 200 mbar. This gives a polyesterol having an acid number of ≦5 1 mg KOH/g.

### Comparative Example 1

894.8 g of phthalic anhydride, 597.35 g of oleic acid, 865.51 g of diethylene glycol and 289.31 g of glycerol are reacted using the general method. This gives a polyesterol having an OH functionality of 2.2 and a hydroxyl number of 259 mg KOH/g.

### Comparative Example 2

953.58 g of phthalic anhydride, 545.65 g of oleic acid, 884.79 g of diethylene glycol and 266.81 g of glycerol are reacted using the general method. This gives a polyesterol having an OH functionality of 2.2 and a hydroxyl number of 237 mg KOH/g.

### Comparative Example 3

A commercially available polyesterol based on dimethyl terephthalate and having a hydroxyl number of 192 mg KOH/g from Invista (Terate 7541 LO) is used.

### Comparative Example 4

1428.51 g of terephthalic acid, 121.46 g of oleic acid, 1460 g of diethylene glycol and 57.69 g of trimethylolpropane are reacted using the general method. This gives a polyesterol having an OH functionality of 2.0 and a hydroxyl number of 228 mg KOH/g.

### Comparative Example 5

1468.53 g of terephthalic acid, 62.43 g of oleic acid, 1500.9 g of diethylene glycol and 40.7 g of glycerol are reacted according to the general method. This gives a polyesterol having an OH functionality of 2.05 and a hydroxyl number of 238 mg KOH/g.

### Example 1

1188.95 g of terephthalic acid, 404.36 g of oleic acid, 1006.3 g of diethylene glycol and 384.12 g of trimethylolpropane were reacted according to the general method. This gave a polyesterol having an OH functionality of 2.3 and a hydroxyl number of 246 mg KOH/g.

### Example 2

1307.33 g of terephthalic acid, 444.57 g of oleic acid, 897.73 g of diethylene glycol and 362.34 g of glycerol were reacted according to the general method. This gave a polyesterol having an OH functionality of 2.5 and a hydroxyl number of 239 mg KOH/g.

The results of the determination of the storage stability are summarized in Table 1.

TABLE 1

| | | | Polyol content with | | | |
|---|---|---|---|---|---|---|
| | | OH number | Fn ≧2.9 (mmol/kg | Appearance | | |
| Polyesterol | Triol | (mg KOH/g) | of PESOL) | 1 month | 2 months | 3 months |
| Comparative Example 4 | Trimethylolpropane | 228 | 172 | Turbid | Turbid | Turbid |
| Comparative Example 5 | Glycerol | 239 | 177 | Turbid | Turbid | Turbid |
| Use Example 1 | Trimethylolpropane | 246 | 1145 | Clear | Clear | Clear |
| Use Example 2 | Glycerol | 239 | 1578 | Clear | Clear | Clear |

Table 1 shows that the polyesterols prepared by the process of the invention are storage-stable for more than 3 months.

### Comparative Examples 6 and 7 and Examples 3 and 4

### Production of Rigid Polyurethane Foams (Variant 1):

The isocyanates and the components which are reactive toward isocyanate were foamed together with the blowing agents, catalysts and all further additives at a constant mixing ratio of polyol component to isocyanate component of 100:190. In each case, a constant fiber time of 49+/−1 seconds and an overall foam density of 33+/−0.5 g/l were set.

### Polyol Component:

79 parts by weight of polyesterol as per Examples 1 and 2 or Comparative Examples 1 and 2

6 parts by weight of polyetherol comprising the ether of ethylene glycol and ethylene oxide having a hydroxyl functionality of 2 and a hydroxyl number of 200 mg KOH/g

13 parts by weight of flame retardant trischloroisopropyl phosphate (TCPP)

2 parts by weight of stabilizer Tegostab B 8443 (silicone-comprising stabilizer)

15 parts by weight of pentane S 80:20

1.5 parts by weight of water

1.6 parts by weight of potassium acetate 47% strength by weight in ethylene glycol)

1.2 parts by weight of 70% bis(2-dimethylaminoethyl)ether

### Isocyanate Component:

190 parts by weight of polymeric MDI (Lupranat® M50 from BASF SE, Ludwigshafen, DE).

The setting of the foam density to 33+/−1 g/l was effected via the water content, and the fiber time was set to 49+/−1 s by varying the bis(2-dimethylaminoethyl) ether content.

The components were foamed with one another as indicated. The curing was determined on the resulting rigid polyurethane foams by means of the indentation test and the flame resistance was measured by determining the flame height as described below.

### Determination of the Curing:

The curing was determined by means of the indentation test. For this purpose, a steel indenter having a hemispherical end having a radius of 10 mm was pressed to a depth of 10 mm into the foam by means of a universal testing machine 2.5, 3, 4, 5, 6 and 7 minutes after mixing of the components in a polystyrene cup. The maximum force required for this in N is a measure of the curing of the foam. As a measure of the brittleness of the rigid polyurethane foam, the point in time at which the surface of the rigid foam had visible fracture zones in the indentation test was determined.

### Determination of the Flame Resistance:

The flame height was measured in accordance with EN ISO 11925-2. The results are shown in Table 2.

TABLE 2

| | Comparative | Comparative | | |
|---|---|---|---|---|
| | Example 6 | Example 7 | Example 3 | Example 4 |
| Polyester polyol | Comparative | Comparative | Example 1 | Example 2 |
| from: | Example 1 | Example 2 | | |
| Indentation test | 39 | 38 | 50 | 53 |
| [N] after 3 min. | | | | |
| Indentation test | 70 | 71 | 84 | 87 |
| [N] after 5 min. | | | | |
| Flame height | 16 | 18 | 11 | 10 |
| [cm] | | | | |

As can be seen from Table 2, the rigid polyurethane foams produced by the process of the invention display improved curing behavior and improved burning behavior.

### Comparative Example 8 and Examples 5 and 6

### Production of Rigid Polyurethane Foams (Variant 2):

The isocyanates and the components which are reactive toward isocyanate were foamed together with the blowing agents, catalysts and all further additives at a constant mixing ratio of polyol component to isocyanate component of 100:190. In each case, a constant fiber time of 49+/−1 seconds and an overall foam density of 41+/−1 g/l were set.

### Polyol Component:

41.5 parts by weight of polyesterol as per Examples 1 and 2 or Comparative Example 2

20 parts by weight of polyetherol having an OHN of ˜490 mg KOH/g and prepared by polyaddition of propylene oxide onto a sucrose/glycerol mixture as starter molecule

6 parts by weight of polyetherol having an OHN of ˜160 mg KOH/g and prepared by polyaddition of propylene oxide onto trimethylolpropane

5 parts by weight of polyetherol comprising the ether of ethylene glycol and ethylene oxide having a hydroxyl functionality of 2 and a hydroxyl number of 200 mg KOH/g

25 parts by weight of flame retardant trischloroisopropyl phosphate (TCPP)

2.5 parts by weight of stabilizer Niax Silicone L 6635 (silicone-comprising stabilizer)

7.5 parts by weight of pentane S 80:20

2.0 parts by weight of water

1.5 parts by weight of potassium acetate (47% strength by weight in ethylene glycol)

0.6 part by weight of a 1:1 mixture of bis(2-dimethylaminoethyl) ether and tetramethylhexanediamine.

### Isocyanate Component:

190 parts by weight of polymeric MDI (Lupranat® M50 from BASF SE, Ludwigshafen, DE)

The setting of the foam density to 41+/−1 g/l was effected via the pentane content and the fiber time was set to 49+/−1 s by varying the proportion of the 1:1 mixture of bis(2-dimethylaminoethyl) ether and tetramethylhexanediamine.

The components A and B were foamed with one another as indicated. The results of the indentation test and the flame heights are shown in Table 3.

TABLE 3

| | Comparative Example 8 | Example 5 | Example 6 |
|---|---|---|---|
| Polyester polyol from: | Comparative Example 2 | Example 1 | Example 2 |
| Indentation test [N] | 53 | 61 | 63 |
| after 3 min. | | | |
| Indentation test [N] | 92 | 101 | 103 |
| after 5 min. | | | |
| Flame height [cm] | 11 | 7 | 10 |

As can be seen from Table 3, the rigid polyurethane foams produced by the process of the invention display improved curing behavior and improved burning behavior.

### Comparative Example 9 and Example 7

In addition, sandwich elements were produced by the double belt process. The foam density was set to 30+/−1 g/I by increasing the water content to 2.6 parts instead of 2 parts and using 11 parts of pentane instead of 7.5 parts. Furthermore, the fiber time was set by varying the proportion of the 1:1 mixture of bis(2-dimethylaminoethyl) ether and tetramethylhexanediamine to 49+/−1 s.

The double belt experiments were carried out using the comparative ester based on dimethyl terephthalate as per Comparative Example 3 and the ester as per Example 1. The assessment of the surface and the processability were determined as described below.

### Determination of the Surface Defects:

The test specimens for assessing the frequency of surface defects were produced by the double belt process.

The surface defects were determined using the above-described method. For this purpose, a 20 cm×30 cm foam specimen was pretreated as described above and illuminated and subsequently photographed. The images of the foam were subsequently digitized and superposed. The integrated area of the black regions of the digital images was divided by the total area of the images so as to give a measure of the frequency of surface defects.

Furthermore, an additional qualitative assessment of the nature of the surface of the rigid polyisocyanurate foams, in which the surface layer of a 1 m×2 m foam specimen was removed and the surfaces were assessed visually with regard to surface defects, was carried out.

### Determination of the Processability:

The processability is determined by examining foam formation during processing. If large bubbles of blowing agents which burst at the foam surface and thus tear this open are formed, these are designated as “blow-outs” and the system cannot be processed in a problem-free manner. If this unsatisfactory behavior is not observed, processing is problem-free.

The results are summarized in Table 4.

TABLE 4

| | Comparative Example 9 | Example 7 |
|---|---|---|
| Polyester polyol from: | Comparative Example 3 | Example 1 |
| Bottom flaws [%]/visual | 16.8%/poor | 4.8%/good |
| assessment | | |
| Processing | blow-outs | problem-free |

Table 4 shows that the rigid polyurethane foams produced by the process of the invention can more easily be produced in a problem-free manner.

### Comparative Examples 10 and 11 and Example 8

### Example

### Production of Rigid Polyurethane Foams (Variant 3):

Furthermore, test plates were produced by the double belt process according to the following production of a rigid polyurethane foam (Variant 3).

The isocyanates and the components which are reactive toward isocyanate were foamed together with the blowing agents, catalysts and all further additives at a constant mixing ratio of polyol component to isocyanate component of 100:170. In each case, a constant fiber time of 28+/−1 seconds and an overall foam density of 37+/−1 g/l were set.

Polyol Component:

58 parts by weight of polyesterol as per Examples or Comparative Examples

10 parts by weight of polyetherol comprising the ether of ethylene glycol and ethylene oxide having a hydroxyl functionality of 2 and a hydroxyl number of 200 mg KOH/g

30 parts by weight of flame retardant trischloroisopropyl phosphate (TCPP)

2 parts by weight of stabilizer Tegostab B 8443 (silicone-comprising stabilizer)

10 parts by weight of n-pentane

1.6 parts by weight of formic acid (85%)

2.0 parts by weight of potassium formate (36% strength by weight in ethylene glycol)

0.6 part by weight of bis(2-dimethylaminoethyl) ether (70% by weight in dipropylene glycol)

### Isocyanate Component:

170 parts by weight of polymeric MDI (Lupranat® M50)

The setting of the foam density to 37 +/−1 g/l was effected via adaptation of the pentane content and the fiber time was set to 28+/−1 s by varying the bis(2-dimethylaminoethyl) ether content.

The components A and B were foamed with one another as indicated. The results of the surface assessment and the processability are summarized in Table 5.

TABLE 5

| | Comparative | Comparative | |
|---|---|---|---|
| | Example 10 | Example 11 | Example 8 |
| Polyester polyol from: | Comparative | Comparative | Example 1 |
| | Example 1 | Example 2 | |
| Bottom flaws [%]/visual | 24.2%/poor | 18.4%/poor | 3.6%/good |
| assessment | | | |
| Processing | blow-outs | blow-outs | problem-free |

Table 5 shows that the rigid polyisocyanurate foams produced by the process of the invention can more easily be produced in a problem-free manner.

1. A polyester polyol, comprising the esterification product of:
a) from 10 to 70 mol % of a dicarboxylic acid composition, comprising
a1) from 50 to 100 mol % of at least one material based on terephthalic acid, selected from the group consisting of terephthalic acid, dimethyl terephthalate, and polyalkylene,
a2) from 0 to 50 mol % of phthalic acid, phthalic anhydride, or isophthalic acid, and
a3) from 0 to 50 mol % of at least one dicarboxylic acid;
b) from 2 to 30 mol % of at least one selected from the group consisting of a fatty acid, a fatty acid derivative, and benzoic acid;
c) from 10 to 70 mol % of at least one aliphatic or cycloaliphatic diol having from 2 to 18 carbon atoms or at least one alkoxylate thereof; and
d) from 2 to 50 mol % of a higher-functional polyol selected from the group consisting of glycerol, alkoxylated glycerol, trimethylolpropane, alkoxylated trimethylolpropane, pentaerythritol, and alkoxylated pentaerythritol,
wherein at least 800 mmol, of polyol d) having an OH functionality of ≧2.9 are reacted per kg of polyester polyol.

2. The polyester polyol of claim 1, wherein the dicarboxylic acid composition a) comprises more than 75 mol % of the material comprising at least one acid a1).

3. The polyester polyol of claim 1, wherein the aliphatic or cycloaliphatic diol c) is selected from the group consisting of ethylene glycol, diethylene glycol, propylene glycol, 1,3-propanediol, 1,4-butanediol, 1,5-pentanediol, 1,6-hexanediol, 2-methyl-1,3-propanediol, and 3-methyl-1,5-pentanediol, and an alkoxylate thereof.

4. The polyester polyol of claim 3, wherein the aliphatic diol is diethylene glycol.

5. The polyester polyol of claim 1, wherein the fatty acid or the fatty acid derivative b2) is a fatty acid or a fatty acid derivative comprising at least one renewable raw material selected from the group consisting of castor oil, a polyhydroxy fatty acid, ricinoleic acid, a hydroxyl-modified oil, grapeseed oil, black cumin oil, pumpkin kernel oil, borage seed oil, soybean oil, wheat germ oil, rapeseed oil, sunflower oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, a hydroxyl-modified fatty acid of myristoleic acid, a hydroxyl-modified fatty acid ester of myristoleic acid, palmitoleic acid, oleic acid, vaccenic acid, petroselic acid, gadoleic acid, erucic acid, nervonic acid, linoleic acid, α-linolenic acid, γ-linolenic acid, stearidonic acid, arachidonic acid, timnodonic acid, clupanodonic acid, and cervonic acid.

6. A process for producing a rigid polyurethane foam, comprising reacting
A) at least one selected from the group consisting of an organic diisocyanate, a modified organic diisocyanate, and a polyisocyanates with
B) at least one polyester polyol of claim 1, wherein the component B) optionally comprises up to 50% by weight of at least one further polyester polyol,
C) optionally, at least one selected from the group consisting of a polyetherol and a further compound having at least two groups which are reactive toward isocyanates and, optionally, at least one of a chain extender and a crosslinker.
D) at least one blowing agent,
E) at least one catalyst,
F) optionally, at least one selected from the group consisting of a further auxiliary and an additive,
G) optionally, at least one flame retardant.

7. A rigid polyurethane foam, obtained by the process of claim 6.

8. (canceled)

9. The polyester polyol of claim 2, wherein the aliphatic or cycloaliphatic diol c) is selected from the group consisting of ethylene glycol, diethylene glycol, propylene glycol, 1,3-propanediol, 1,4-butanediol, 1,5- pentanediol, 1,6-hexanediol, 2-methyl-1,3-propanediol, and 3-methyl-1,5- pentanediol, and an alkoxylate thereof.

10. The polyester polyol of claim 1, wherein the aliphatic diol c) is diethylene glycol.

11. The polyester polyol of claim 1, wherein an amount of the dicarboxylic acid composition a), from which the esterification product is formed, is from 20 to 70 mol %

12. The polyester polyol of claim 1, wherein an amount of the dicarboxylic acid composition a), from which the esterification product is formed, is from 25 to 50 mol %
